(19) United States
(12) Patent Application Publication
(10) Pub. No.: US 2004/0204658 A1
(43) Pub. Date: Oct. 14, 2004

(54) Systems and methods for providing an enhanced bioelectric sensing surface

(76) Inventors: Phillip W. Dietz, St. George, UT (US); Douglas S. Horne, Murray, UT (US)

Correspondence Address: Michael F. Krieger, Kirton & McConkie, 60 East South Temple, Suite 1800, Salt Lake City, UT, 84111, US

(21) Appl. No.: 10/411,030

(22) Filed: Apr. 10, 2003

(51) Int. Cl. A61B 5/05

(52) U.S. Cl. 600/547

(57) ABSTRACT

Systems and methods for providing and using an enhanced bioelectric sensing surface to facilitate locating and obtaining a bioelectric resistance value from a patient for therapeutic and/or diagnostic purposes. In one implementation, a bioelectric probe tip includes a conductive base having a configuration. An abrasive bristly conductive surface is coupled to or otherwise provided on a surface area of the conductive base, wherein the abrasive bristly conductive surface includes a plurality of bristles. Multiple bristles are able to simultaneously contact a surface layer of a patient's skin to enable the bioelectric probe tip to locate and obtain a bioelectric resistance value from the patient.

### BACKGROUND OF THE INVENTION

1. Field of the Invention

The present invention relates to obtaining bioelectric information. In particular, the present invention relates to systems and methods for providing and using an enhanced surface to facilitate locating and obtaining a bioelectric resistance value from a patient for assessment, therapeutic and/or diagnostic purposes.

2. Background and Related Art

Traditional medical science has long recognized certain electrical characteristics of humans and other living organisms. For example, the traditional medical community has recognized the electrical potentials generated by the human body in such forms as brain waves as detected by electro-encephalographs (EEG), electrical impulses resulting from muscular heart activity as detected by electrocardiograms (EKG), and other electrical potentials measurable at other areas of the human body. While the relative levels of the electrical activity exhibit relatively small levels, such signals are nonetheless measurable and consistent.

In addition to measurable voltage levels, the human body and other mammalian organisms exhibit specific locations wherein the resistance value and the conductance value are relatively predictable for healthy individuals. The locations of anatomical dermal conductance points exhibit unique resistance values. Interestingly, the locations on the body exhibit a resistive reading of approximately 100,000 ohms and coincide with the body locations that correspond to the acupuncture points defined anciently by the Chinese. Indeed, Chinese medical practitioners were aware of the art of treating unfavorable health conditions through the use of needles that are used to pierce peripheral nerves to relieve pain. Electrical stimulation of these points provides similar results. Many acupuncture points are situated above major nerve trunks and have nerves within 0.5 centimeters of their location. Studies have indicated that many acupuncture points correspond to nerve innervations and trigger points. The acupuncture points are located under the skin (epidermis) and are accessed electrically through the skin either by the use of acupuncture needles or by using a probe tip pressed against the skin. As the outermost layer of epidermis (cornified layer) is less conductive, the probe tip may or may not need a fluid or a type of electrode gel to enhance conductivity through the skin to the acupuncture point. Even so, the amount of pressure required to access the point can frequently invoke painful responses from the patient.

The representative acupuncture points and their relationship with organs and life systems of the human body have been characterized into more than 800 points that are organized into approximately 14 basic meridians. The measurable state of these acupuncture points reflects the condition of the related meridians and therefore the health of organs and other functions of the human body.

In the art of acupuncture, the acupuncture points are generally located at the extremity region of the hands and feet. As introduced above, the resistance value of healthy tissue at an acupuncture or conductance point is generally in the range of about 100,000 ohms. When such tissue is inflamed or infected, the conductivity is higher such that the measured resistance value appears lower than 100,000 ohms. Additionally, if the tissue is in a degenerative state, the conductivity is lower causing the resistance value to be higher.

Systems have been implemented to measure the resistance value at acupuncture points and present resistive values to a clinician for use in diagnosing a condition. However, the traditional systems have proven difficult to use since the precise location of the points is difficult to pinpoint, often requiring a probe tip to be placed on a specific angle in relation with the surface of the patient. Further, the differences in the characteristics of each patient and each point of a given patient can cause a practitioner to obtain inaccurate and/or unrepeatable readings. Moreover, current technologies have caused pain and/or discomfort to patients.

In some systems, a first device is used to locate the points and a second device is brought in contact with the point to perform the electro-dermal screening. While this technique is available, employing multiple devices introduces a potential for clinical error. Accordingly, other systems have been made available that include both a point finding function and an electro-dermal screening function. However, in every case the system used proves difficult to locate the points on the patient. And, the electro-dermal screening is compromised when the system does not accurately determine the points.

Thus, while techniques currently exist that are used to locate a point on a patient, challenges still exist, such as inaccurate readings, unrepeatable readings, pain, discomfort, and the like. Accordingly, it would be an improvement in the art to augment or even replace current techniques with other techniques.

### SUMMARY OF THE INVENTION

The present invention relates to obtaining bioelectric information. In particular, the present invention relates to systems and methods for providing and using an enhanced surface to facilitate locating and obtaining a bioelectric resistance value from a patient for assessment, therapeutic and/or diagnostic purposes.

Implementation of the present invention takes place in association with an abrasive bristly conductive surface that is used to obtain a bioelectric value from a patient. Multiple bristles of the abrasive bristly conductive surface are able to simultaneously contact and/or puncture a surface layer of a patient's skin (e.g., the cornified layer of the epidermis) to enable and facilitate locating and obtaining a bioelectric resistance value from the patient.

In one implementation, the abrasive bristly conductive surface is used in association with a bioelectric probe tip that includes a conductive base having a configuration. The abrasive bristly conductive surface is obtained by coupling a conductive bristly matrix to a surface area of the conductive base, wherein the abrasive bristly matrix includes a plurality of bristles.

In another implementation, the abrasive bristly conductive surface is used in association with a bioelectric probe tip that includes a non-conductive base having a configuration. The abrasive bristly conductive surface is obtained by coupling a conductive bristly matrix to a surface area of the non-conductive base, wherein the abrasive bristly matrix includes a plurality of bristles. In other implementations, the abrasive bristly conductive surface is machined, molded, etched or otherwise provided into the base, wherein the abrasive bristly matrix includes a plurality of bristles.

In at least one implementation of the present invention, the bioelectric probe tip includes a convex distal end having the abrasive bristly conductive surface. In this implementation, the convex distal end may be wider than traditional probe tips to facilitate location of the point. Moreover, the convex nature along with the abrasive bristly conductive surface reduces the requirement of using the probe on a particular angle in relation to the patient. In addition, the bristles of the distal end may be used to penetrate the cornified layer of the epidermis without puncturing the epidermis and to access the conductive layer with less pressure than traditional probes. The reduction of the pressure of the probe greatly increases the patient's comfort while the bioelectric resistance readings are taken.

While the methods and processes of the present invention have proven to be particularly useful in association with a bioelectric probe, those skilled in the art will appreciate that the methods and processes can be used in association with a variety of different bioelectric sensing devices, including patches, clips, and the like to provide an enhanced bioelectric sensing surface. Moreover, the methods and processes of the present invention embrace a variety of different configurations other than a convex distal end of a bioelectric probe tip.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a representative base configuration for a bioelectric probe tip in accordance with an embodiment of the present invention;

FIG. 2 illustrates another representative base configuration for a bioelectric probe tip in accordance with an embodiment of the present invention;

FIG. 3 illustrates another representative base configuration for a bioelectric probe tip in accordance with an embodiment of the present invention;

FIG. 4 illustrates another representative base configuration for a bioelectric probe tip in accordance with an embodiment of the present invention;

FIG. 5 illustrates another representative base configuration for a bioelectric probe tip in accordance with an embodiment of the present invention;

FIG. 6 illustrates a first end view of the base configuration of FIG. 5;

FIG. 7 illustrates a second end view of the base configuration of FIG. 5;

FIG. 8 illustrates a cross-sectional view of the base configuration of FIG. 5;

FIG. 9 illustrates a perspective view of the base configuration of FIG. 5;

FIG. 10 illustrates a illustrates the base configuration of FIG. 5 having an abrasive bristly conductive surface;

FIG. 11 illustrates use of a base configuration having an abrasive bristly conductive surface for use in locating and obtaining a bioelectrical resistance value from a patient;

FIG. 12 illustrates another representative embodiment of the present invention, wherein an abrasive bristly conductive surface is provided by being machined, etched, molded or otherwise manufactured;

FIG. 13 illustrates a magnified view of the abrasive bristly conductive surface of FIG. 12;

FIG. 14 illustrates a front perspective view of another representative embodiment of the present invention; and

FIG. 15 illustrates a back perspective view of the bioelectric patch of FIG. 14.

### DETAILED DESCRIPTION OF THE INVENTION

The present invention relates to obtaining bioelectric information. In particular, the present invention relates to systems and methods for providing and using an enhanced surface that is used to facilitate locating and obtaining a bioelectric resistance value from a patient for assessment, therapeutic and/or diagnostic purposes.

Embodiments of the present invention take place in association with an abrasive bristly conductive surface that is used to obtain a bioelectric value. Some embodiments embrace a bioelectric probe having a tip that includes a base with a configuration or shape, and wherein the abrasive bristly conductive surface is coupled, machined, molded and/or manufactured to a surface area of the base. The abrasive bristly conductive surface includes a plurality of bristles such that a variety of the bristles are able to simultaneously contact and/or puncture the cornified layer of a patient's epidermis to enable at least one bristle to be in contact with the acupuncture point and to obtain a bioelectric resistance value from the patient. Other embodiments of the present invention embrace an abrasive bristly conductive surface used in association with other biometric sensing devices, such as patches, clips, and the like.

In the disclosure and in the claims the term “abrasive bristly conductive surface” shall refer to an abrasive construction of a plurality of peaks/bristles, wherein at least one of the peaks/bristles is able to locate and/or obtain a bioelectric value of a patient. An example of an abrasive bristly conductive surface includes one or more materials and/or coatings such as: metallic carbide, tungsten, silver, nickel, brass, copper, gold, aluminum, any other metal or metal alloy, and/or any other conductive material, including graphite, any electrical conductor, any ionic conductor, any conducting polymer, and the like.

In accordance with at least some embodiments of the present invention, a patient may undergo bioelectric therapy corresponding to a condition diagnosed at an anatomical dermal conductance point. The various anatomical dermal conductance points are typically located throughout a patient's hands and feet. The dermal conductance points or acupuncture points aid the clinician in assessing and/or diagnosing a patient's condition and pinpointing a particular disorder. The dermal conductance points are typically about 1-3 mm in diameter and are located just under the epidermal layer near the neck of the bones of the hands and feet.

In accordance with embodiments of the present invention, a patent's condition may be assessed and/or diagnosed using a device or equipment capable of measuring the resistance or likewise the conductance at anatomical dermal conductance points located throughout the hands and feet of the patient. By way of example, representative systems used to evaluate and diagnose a condition of a patient include bioelectric probes, bioelectric patches, bioelectric clips, and the like having an abrasive bristly conductive surface and used in association with an anatomical dermal conductance point.

Thus, while embodiments of the present invention embrace a variety of different systems having an abrasive bristly conductive surface, the following relates to a representative system that includes a bioelectric probe having a probe tip. The probe tip is placed on an anatomical dermal conductance point. The conductance value is measured between the probe and a ground bar, and is displayed on a conductance monitor or other output for evaluation by a clinician or practitioner. If the conductance value at a particular conductance point on the patient denotes an imbalance, the clinician may investigate the biological system meridian that corresponds to the conductance point presenting the imbalanced reading. Conversely, when a particular conductance point displays a balanced reading, the clinician thereafter measures the conductance at various other conductance points to properly assess and/or diagnose the condition of the patient.

Upon evaluating the condition of the patient, such as an organ disorder or a biological system abnormality, the clinician selects a possible remedy for such a condition. Remedies may include providing a homeopathic remedy or a digital sequence that is known to exhibit a particular reaction on an individual. An electromagnetic energy source generates a frequency coded electromagnetic signal containing the digital sequence, which is broadcast or projected upon the patient. The frequency coded electromagnetic signal may take the form of several electromagnetic types, such as radio frequency (RF) signals, infrared (IR) signals or other electromagnetic projections.

Accordingly, as provided herein, embodiments of the present invention utilize an abrasive bristly conductive surface in association for use in obtaining a bioelectric resistance value. In some embodiments, a bioelectric probe tip is configured with the abrasive bristly conductive surface to obtain the bioelectrical resistance value. In particular, the enhanced bioelectric probe tip includes a conductive or non-conductive base having a configuration and an abrasive bristly conductive surface that is coupled to, machined on, molded with and/or manufactured onto a surface area of the base, wherein the abrasive bristly conductive surface includes a plurality of bristles that simultaneously contact and/or penetrate a surface layer of a patient's skin (e.g., the cornified layer of the epidermis) to enable the abrasive bristly conductive surface to locate and obtain a bioelectric resistance value from the patient, as will be further discussed below.

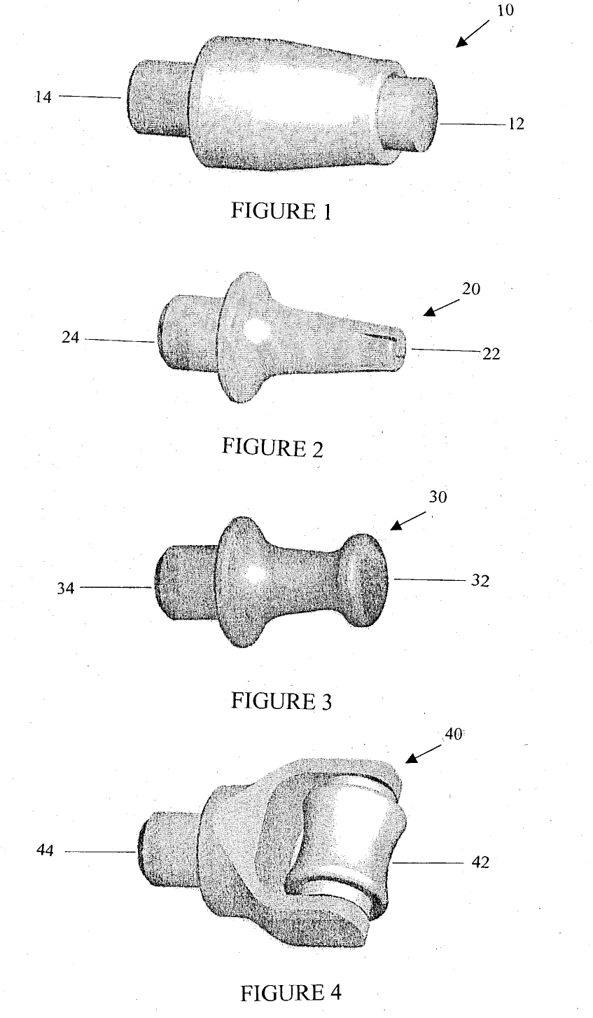

With reference now to FIG. 1, a representative conductive or non-conductive base having a configuration for use as a bioelectric probe tip is illustrated in accordance with an embodiment of the present invention. In FIG. 1, base configuration 10 comprises a conductive material or non-conductive material that is coated, plated or manufactured to have a surface of conductive material. Examples of conductive materials include metallic materials, including stainless steel, brass, silver, nickel, copper, gold, graphite and other metals, alloys and/or any other conductive materials, including electrical conductors, ionic conductors, etc.

As illustrated, base configuration 10 includes a distal end 12 and a proximal end 14. As will be further discussed below, an abrasive bristly conductive surface (not shown) is coupled, machined, etched, molded and/or manufactured to at least a portion of distal end 12 and is used to locate and/or obtain a bioelectrical resistance value from the patient. Distal end 12 is convex and may be wider than traditional probe tips to facilitate location of the conductance point. The convex nature of distal end 12 reduces the need for a precise location and/or a precise angle in relation with the dermal surface layer to locate and obtain the bioelectric resistance value. Proximal end 14 is configured for electronic coupling to a mechanism for reading the bioelectrical resistance value.

While FIG. 1 illustrates a particular tip configuration, those skilled in the art will appreciate that the methods and processes of the present invention can be used in association with a variety of different tip/base configurations to provide an enhanced bioelectric sensing surface. Accordingly, FIGS. 2-5 provide additional representative tip/base configurations for use in association with the present invention. Those skilled in the art will appreciate that the base configurations provided herein are illustrative only, and that the embodiments of the present invention embrace a variety of other types of base configurations onto which an abrasive bristly conductive surface may be coupled, machined, etched, molded and/or otherwise manufactured for use in locating and/or obtaining a bioelectric resistance value from the patient.

With reference to FIG. 2, another representative conductive or non-conductive base having a configuration for use as a bioelectric probe tip is illustrated in accordance with an embodiment of the present invention. In FIG. 2, base configuration 20 comprises a conductive or non-conductive material, and includes a distal end 22 and a proximal end 24. As will be further discussed below, an abrasive bristly conductive surface (not shown) is coupled, machined, etched, molded and/or manufactured to at least a portion of distal end 22 for use in locating and/or obtaining a bioelectrical resistance value from the patient. Proximal end 24 is configured for electronic coupling to a mechanism for reading the bioelectrical resistance value.

In FIG. 3, another representative base having a configuration for use as a bioelectric probe tip is illustrated as base configuration 30. Base configuration 30 comprises a conductive or non-conductive material, and includes a distal end 32 and a proximal end 34. An abrasive bristly conductive surface (not shown) is coupled, machined, etched, molded and/or manufactured to at least a portion of distal end 32 and is used to locate and/or obtain a bioelectrical resistance value from the patient. Distal end 32 is convex and may be wider than traditional probe tips to facilitate location of conductance points. The convex nature of distal end 32 reduces the need for a precise location and/or a precise angle in relation with the dermal surface layer to locate and obtain the bioelectric resistance value. Proximal end 34 is configured for electronic coupling to a mechanism for reading the bioelectrical resistance value.

With reference now to FIG. 4, another representative conductive base having a configuration for use as a bioelectric probe tip is illustrated in accordance with an embodiment of the present invention. In FIG. 4, base configuration 40 comprises a conductive or non-conductive material, and includes a distal end 42 and a proximal end 44. An abrasive bristly conductive surface (not shown) is coupled, machined, etched, molded and/or manufactured to at least a portion of distal end 42 and is used to locate and/or obtain a bioelectrical resistance value from the patient. Distal end 42 includes a roller. Furthermore, distal end 42 is concave to reduce the requirement of using the probe on a particular angle in relation to the patient. Proximal end 44 is configured for electronic coupling to a mechanism for reading the bioelectrical resistance value.

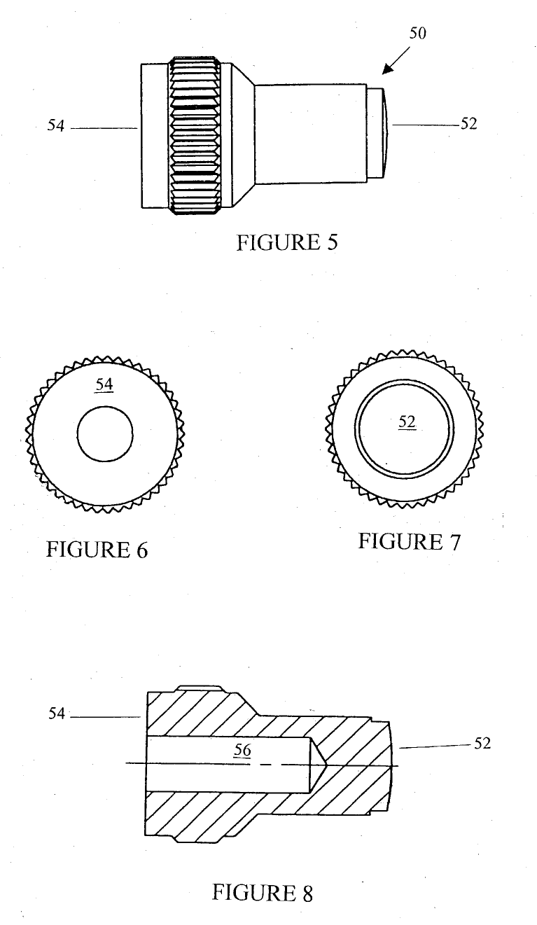

With reference now to FIG. 5, another representative conductive base having a configuration for use as a bioelectric probe tip is illustrated. In FIG. 5, base configuration 50 comprises a conductive or non-conductive material, which may be coated, plated or manufactured to have a surface of conductive material. In one embodiment, base configuration comprises stainless steel. As illustrated, base configuration 50 includes distal end 52 and proximal end 54. An abrasive bristly conductive surface (not shown) is coupled, machined, molded and/or manufactured to at least a portion of distal end 52 and is used to locate and/or obtain a bioelectrical resistance value from the patient. Distal end 52 is convex and may be wider than traditional probe tips to facilitate location of the conductance point. The convex nature of distal end 52 reduces the need for a precise location and/or a precise angle in relation with the dermal surface layer to locate and obtain the bioelectric resistance value. Proximal end 54 is configured for electronic coupling to a mechanism for reading the bioelectrical resistance value. A planar view of proximal end 54 and distal end 52 are respectively illustrated in FIGS. 6 and 7.

With reference now to FIG. 8, a cross-sectional view of base configuration 50 of FIG. 5 is illustrated, having distal end 52 and proximal end 54. In a further embodiment, configuration 50 is machined from magnetic stainless steel. However, those skilled in the art will appreciate that other conductive materials may be used. Moreover, those skilled in the art will appreciate that other embodiments embrace one or more non-conductive materials that are coated, plated or manufactured to have a surface of conductive material. In the illustrated embodiment, configuration 50 includes channel 56, which is configured to receive a threaded insert and circuitry to take an isolated reading.

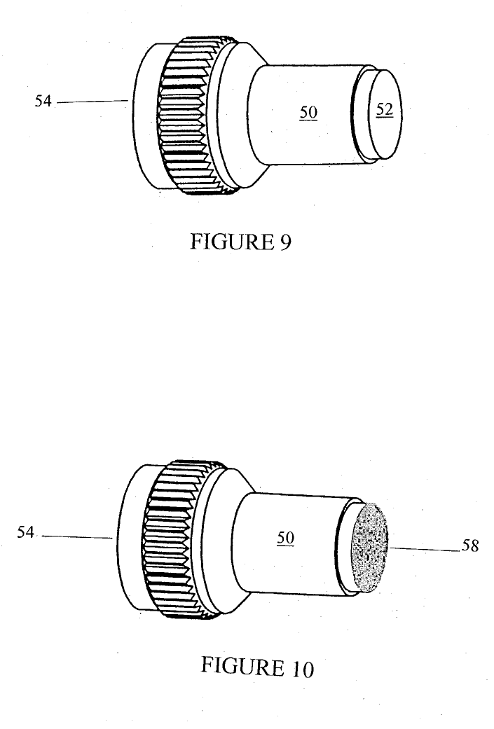

As provided herein, an abrasive bristly conductive surface is coupled, machined, etched, molded and/or manufactured to a surface area of at least a portion of a base configuration. This is illustrated in FIGS. 9-10, wherein FIG. 9 illustrates base configuration 50 of FIG. 5 prior to the inclusion of an abrasive bristly conductive surface, and wherein FIG. 10 illustrates base configuration 50 of FIG. 5 with an abrasive bristly conductive surface 58 coupled, machined, etched, molded and/or otherwise manufactured to the distal end of configuration 50.

While an abrasive bristly conductive surface may be coupled, machined, etched, molded and/or otherwise manufactured to a base configuration in a variety of manners, in one embodiment a magnetic field is used to couple an abrasive bristly matrix (e.g., matrix 58) to a portion of a base configuration (e.g., distal end 52). In particular, base configuration 50 is placed into a magnetic field to attract metallic pieces/filings (e.g., particles of tungsten, silver, nickel, brass, copper, gold, aluminum, any other metal or metal alloy, and/or any other conductive material). The magnetic field causes the metallic pieces to stand on end at distal end 52, which is then heated to braise the metallic pieces onto distal end 52 of configuration 50. Optionally, in some embodiments, the configuration is shaped or otherwise machined after the sintering process. In another embodiment, a conductive material (e.g., graphite) is combined with a binder and adhered to the base. Moreover, a coating (e.g., silver plate, brass plate, gold plate, nickel plate or other coating or a combination of coatings or platings) is optionally applied after the abrasive bristly matrix is coupled, machined, etched, molded and/or manufactured to the base configuration.

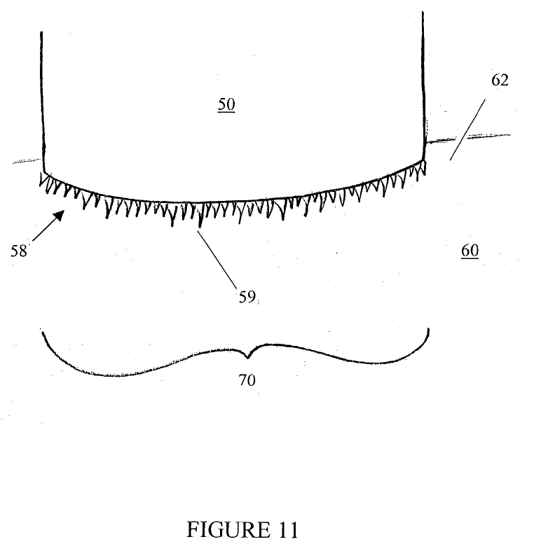

Accordingly, with reference now to FIG. 11, the configuration 50 with matrix 58 may be used as a bioelectric probe tip in locating and/or obtaining a bioelectric resistance value from a patient. In particular, matrix 58 includes a plurality of bristles 59, wherein one or more of bristles 59 are able to locate and/or obtain the bioelectric resistance value. Thus, the bioelectric probe tip may be selectively used to contact a dermal surface layer 62 of a patient 60 to locate and/or obtain a bioelectric value of the patient 60. The plurality of bristles 59 that are able to obtain the bioelectric value enables a greater surface coverage 70 of dermal surface layer 62, thereby facilitating locating and obtaining the bioelectric value of the patient 60.

In some embodiments, the bristles are random in length and/or are consistently located on at least a portion (e.g., distal end 52) of base configuration 50. In other embodiments, the bristles are more uniform. Further, in some embodiments one or more of the bristles puncture the cornified layer of the epidermis to obtain the bioelectric value(s). In other embodiments, bristles do not puncture the cornified layer and may optionally be use in combination with a material, such as water, to enhance obtaining the bioelectric value(s).

As provided herein, embodiments of the present invention embrace a variety of techniques for providing an abrasive bristly conductive surface that may be used to obtain bioelectric readings. For example, the abrasive bristly conductive surface may be etched (e.g., mechanically and/or chemically), machined, molded, or otherwise provided.

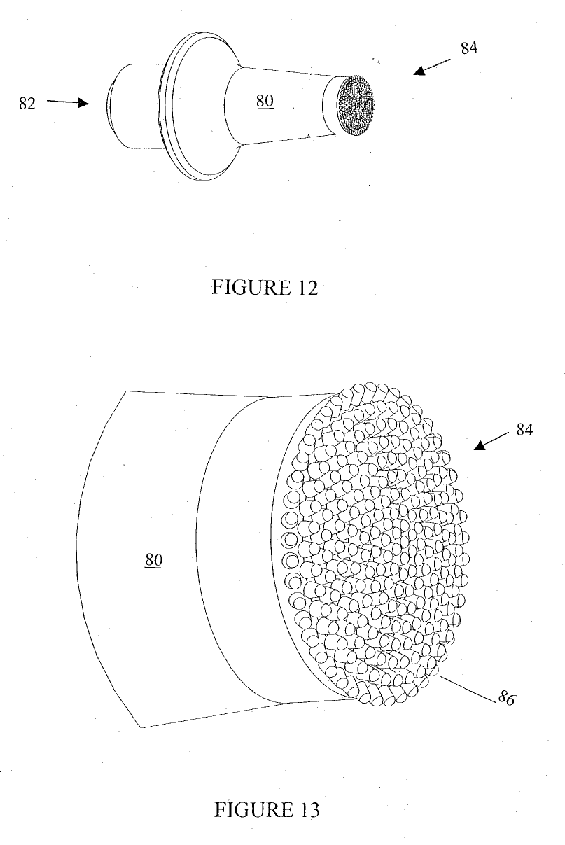

With reference now to FIGS. 12-13, an embodiment of the present invention is illustrated as tip 80 having a proximal end 82 configured for coupling with a bioelectric probe and an abrasive bristly conductive surface 84, having a plurality of bristles 86 (FIG. 13), configured to obtain bioelectric values. Tip 80 is manufactured by being machined or cast/molded.

Further, in accordance with some embodiments of the present invention, a non-conductive base is manufactured, machined, cast/molded, etched, or otherwise provided having an abrasive bristly surface that is plated with a conductive material.

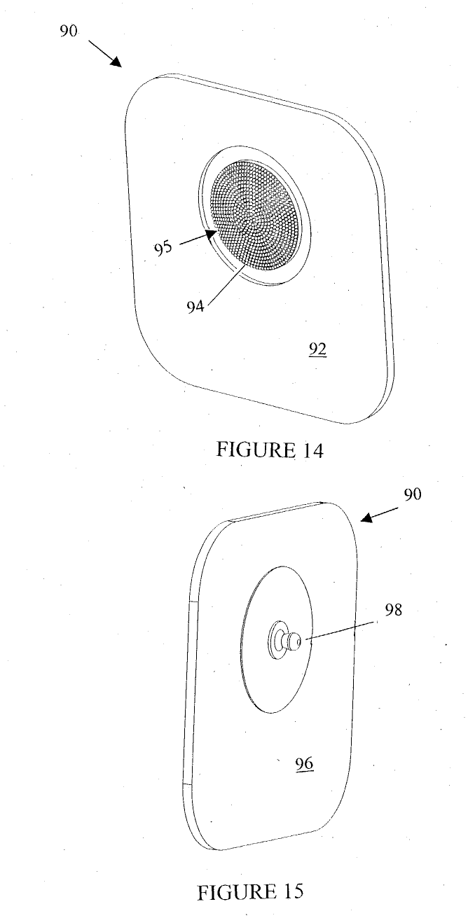

Moreover, while the representative embodiments provided herein have related to bioelectric probe tips, embodiments of the present invention also embrace the use of an abrasive bristly conductive surface in association with other devices (e.g., one or more patches, clips, etc.) to obtain bioelectric values. For example, with reference now to FIGS. 14-15, a bioelectric patch is provided as bioelectric patch 90. In FIG. 14, a front surface 92 of patch 90 is provided that includes a plurality of bristles 94 to provide an abrasive bristly conductive surface 95. As provided herein, bristles 94 may be coupled, machined, etched, molded and/or manufactured to at least a portion of patch 90. Front surface 92 is configured to be applied/adhered to a patient. FIG. 15 illustrates an opposing surface 96 of patch 90 and includes a contact point 98 for the connection of a wire or electrical lead thereto. Patch 90 is selectively applied to a patient and the abrasive bristly conductive surface is used to obtain a bioelectric value from the patient.

Those skilled in the art will appreciate that while the abrasive bristly conductive surface has been illustrated in a circular configuration, other embodiments embrace other non-circular configurations of an abrasive bristly conductive surface. Further, as provided herein, at least some of the embodiments of the present invention include an abrasive bristly conductive surface that includes a high density of bristles rather than just having multiple points to enable an enhanced bioelectric sensing surface.

Thus, as discussed herein, embodiments of the present invention embrace obtaining bioelectric information. In particular, embodiments of the present invention relate to systems and methods for providing and using an enhanced surface to facilitate locating and obtaining a bioelectric resistance value from a patient for therapeutic and/or diagnostic purposes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A bioelectric sensing surface configured to obtain a bioelectric resistance value of a patient, the sensing surface comprising:
an abrasive bristly conductive surface that includes a high density of bristles, wherein the abrasive bristly conductive surface is configured such that a plurality of bristles of the abrasive bristly conductive surface simultaneously contact a dermal surface layer of a patient to enable the sensing surface to locate and obtain a bioelectric resistance value from the patient.

2. A bioelectric sensing surface as recited in claim 1, wherein the abrasive bristly conductive surface is located on at least a portion of a base having a configuration.

3. A bioelectric sensing surface as recited in claim 2, wherein the base is one of:
(i) a bioelectric probe tip;
(ii) a bioelectric patch; and
(iii) a bioelectric clip.

4. A bioelectric sensing surface as recited in claim 2, wherein one of (i) a machining process, (ii) an etching process, (iii) a casting process, (iv) a molding process, and (v) an adhering process locates the abrasive bristly conductive surface on the portion of the base.

5. A bioelectric sensing surface as recited in claim 4, wherein the etching process is one of:
(i) a mechanical etching process; and
(ii) a chemical etching process.

6. A bioelectric sensing surface as recited in claim 2, wherein the base comprises one of:
(i) a conductive material; and
(ii) a non-conductive material.

7. A bioelectric sensing surface as recited in claim 2, wherein the configuration includes at least one of:
(i) a convex surface;
(ii) a concave surface;
(iii) a flat surface.

8. A bioelectric sensing surface as recited in claim 7, wherein the configuration further includes at least one of:
(i) a wide distal end; and
(ii) a roller.

9. A bioelectric sensing surface as recited in claim 1, wherein the abrasive bristly conductive surface is a carbide.

10. A bioelectric sensing surface as recited in claim 1, wherein the abrasive bristly conductive surface comprises at least one of:
(i) a metal;
(ii) a metal alloy;
(iii) graphite;
(iv) an electrical conductor;
(v) an ionic conductor; and
(vi) a conducting polymer.

11. A bioelectric sensing surface as recited in claim 1, wherein the abrasive bristly matrix reduces the need for at least one of: (i) a precise location and (ii) a precise angle in relation with the dermal surface layer to locate and obtain the bioelectric resistance value.

12. A method for manufacturing a device for use in obtaining a bioelectric resistance value from a patient, the method comprising:
providing a base having a configuration;
forming an abrasive bristly conductive surface on at least a portion of the base, wherein the abrasive bristly conductive surface is configured to contact a dermal surface layer of a patient, and wherein the abrasive bristly conductive surface is further configured to locate and obtain a bioelectric resistance value of the patient.

13. A method as recited in claim 12, wherein the base is a portion of one of:
(i) a bioelectric probe tip;
(ii) a bioelectric patch; and
(iii) a bioelectric clip.

14. A method as recited in claim 12, wherein the step for forming an abrasive bristly conductive surface comprises at least one of the steps for:
(i) machining the abrasive bristly conductive surface on the base:
(ii) etching the abrasive bristly conductive surface on the base;
(iii) casting the abrasive bristly conductive surface;
(iv) molding the abrasive bristly conductive surface; and
(v) adhering the abrasive bristly conductive surface on the base.

15. A method as recited in claim 12, wherein the step for forming an abrasive bristly conductive surface includes the steps for;
providing an abrasive bristly surface on the base; and
coating the abrasive bristly surface with a conductive coating.

16. A bioelectric probe tip configured to obtain a bioelectric resistance value of a patient, the probe tip comprising:
a base having a configuration; and
an abrasive bristly conductive surface located on a surface area of the base, wherein the bioelectric probe tip is configured such that a plurality of bristles of the abrasive bristly conductive surface simultaneously contact a dermal surface layer of a patient to enable the tip to locate and obtain a bioelectric resistance value from the patient.

17. A bioelectric probe tip as recited in claim 16, wherein the abrasive bristly matrix reduces the need for at least one of:
(i) a precise location to locate and obtain the bioelectric resistance value; and
(ii) a precise angle in relation with the dermal surface layer.

18. A bioelectric probe tip as recited in claim 16, wherein the base comprises a conductive material.

19. A bioelectric probe tip as recited in claim 16, wherein the configuration includes at least one of:
(i) a convex surface;
(ii) a concave surface;
(iii) a flat surface;
(iv) a wide distal end; and
(v) a roller.

20. A bioelectric probe tip as recited in claim 16, wherein at least one of (i) the base and (ii) the abrasive bristly conductive surface includes a conductive coating.
